(19) United States
(12) Patent Application Publication
(10) Pub. No.: US 2002/0082237 A1
(43) Pub. Date: Jun. 27, 2002

(54) Cationic polymers and lipids for the delivery of nucleic acids

(76) Inventors: Sean M. Sullivan, The Woodlands, TX (US); Xiao-Ying Meng, Mountain View, CA (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/084,159

(22) Filed: Feb. 28, 2002

Related U.S. Application Data
(63) Continuation of application No. 08/865,375, filed on May 29, 1997, now abandoned.
(60) Provisional application No. 60/018,377, filed on May 29, 1996.

(51) Int. Cl. A61K 48/00; C07J 9/00; C12N 15/88

(52) U.S. Cl. 514/44; 435/458; 528/288; 552/540

(57) ABSTRACT

Novel cationic polymers and cationic lipids, and methods of making and using the same, are provided. The cationic polymers and cationic lipids are useful for the delivery of nucleic acid polymers and oligomers to cells in vitro and in vivo.

### CROSS REFERENCE TO RELATED APPLICATIONS

The present application claims priority to U.S. provisional Application Ser. No. 60/018,377, filed May 29, 1996.

### 1.0. INTRODUCTION

The present invention is in the field of biochemistry. In particular, novel compositions and methods are reported which efficiently deliver polynucleotides or other bioactive materials to cells.

### 2.0. BACKGROUND

The present invention relates to novel cationic polymers and cationic lipids that are useful for the delivery of polynucleotides to cells. As the field of molecular biology has matured, a wide variety of methods and techniques have evolved which allow researchers to engineer polynucleotides. Polynucleotides are typically engineered to perform a specific function within the cell. Unfortunately, polynucleotide polymers are highly charged molecules (due to the phosphate backbone) and do not readily permeate the cell membrane. As such, concomitant with the advances made in genetic engineering, advances have also been made in methods by which researchers may introduce genetically engineered material into cells.

One of the methods developed for delivering genetically engineered polynucleotides to cells involves the use of liposomes. The phospholipid bilayer of the liposome is typically made of materials similar to the components of the cell membrane. Thus, polynucleotides associated with liposomes (either externally or internally) may be delivered to the cell when the liposomal envelope fuses with the cell membrane. More typically, the liposome will be endocytosed into the cell. After internalization, the internal pH of the endocytic vesicle may drop substantially, and/or the vesicle may fuse with other intracellular vesicles, including lysosomes. During or subsequent to the process of vesicle fusion, the internal contents of the endosome may be released into the cell.

Liposomes are limited as polynucleotide delivery vehicles by the relatively small internal volume of the liposome. Thus, it is difficult to effectively entrap a large concentration of polynucleotide within a liposomal formulation.

Researchers have tried to compensate for the above inefficiency by adding or using positively charged amphipathic lipid moieties to liposomal formulations. In principle, the positively charged groups of the amphipathic lipids will ion-pair with the negatively charged polynucleotides and increase the amount-of association between the polynucleotides and the lipidic particles. The enhanced association presumably promotes binding of the nucleic acid to the lipid bilayer. For example, several cationic lipid products are currently available which are useful for the introduction of nucleic acid into the cell. Particularly of interest are, LIPOFECTIN™ (DOTMA) which consists of a monocationic choline head group which is attached to diacylglycerol (see generally, U.S. Pat. No. 5,208,036 to Epstein et al.); TRANSFECTAM™ (DOGS) a synthetic cationic lipid with lipospermine head groups (Promega, Madison, Wis.); DMRIE and DMRIE•HP (Vical, La Jolla, Calif.); DOTAP™ (Boehringer Mannheim (Indianapolis, Ind.), and Lipofectamine (DOSPA) (Life Technology, Inc., Gaithersburg, Md.).

Properly employed, the above compounds enhance the permeability of nucleic acids to cells cultured in vitro, and the process of lipofection has become an important tool of cellular biology. Typically, formulations comprising the cationic lipids are intermixed with the polynucleotide to be delivered and then applied to the target cells. The cationic lipid-polynucleotide complex must generally be used relatively soon after mixing because lipofection efficiency rapidly decreases to undetectable levels 24 hours after formulation. From this observation, one may surmise that, at least with respect to lipofection efficiency, the cationic lipid-polynucleotide complex is rather unstable, or has an extremely limited shelf life.

From a research perspective, the above complexes are rather facile to prepare. Thus, the relatively short active-life of the prepared complex does not hinder analytical applications in vitro. However, where the medical or in vivo use of polynucleotide delivery vehicles comprising cationic lipids is contemplated, it would be desirable to remove the uncertainty added by entrusting product formulation (as opposed to mere reconstitution) to the medical clinician. This becomes even more apparent when one considers that the formulations must be used within a narrow window of optimum activity. Thus, particularly where clinical use is contemplated, a more stable polynucleotide delivery system would be preferred.

Another draw-back of the presently available synthetic cationic lipids is that the respective lipid and cationic components are not joined by a biodegradable chemical linkage which presumably contributes to the inherent toxicity of the synthetic cationic lipids.

A given level of cellular toxicity may be detrimental but acceptable where in vitro, or research, use of cationic lipids to deliver polynucleotides is contemplated; however, such toxicity is generally unacceptable where in vivo use of cationic lipids is contemplated. Thus, synthetic cationic lipids which comprise biocompatible, biodegradable, or metabolizable components would be preferred for the preparation of cationic lipid-polynucleotide delivery vehicles for use in vivo.

Alternatively, other lipid groups may be joined to suitable cationic components in an attempt produce cationic lipids with reduced toxicity.

Additionally, the toxicity of the synthetic cationic lipids may be reduced by assembling the cationic lipids into suitably constructed polynucleotide delivery vehicles.

Finally, the currently available methods for using synthetic cationic lipids to transfect cells all produce lipid/DNA complexes which are rapidly inactivated by relatively low concentrations of serum. Serum sensitivity may be easily circumvented in in vitro applications by conducting the initial portions of the transfection procedure in serum free medium. However, serum sensitivity remains a major obstacle to the wide-spread use of cationic lipid-mediated DNA delivery in vivo.

### 3.0 SUMMARY OF THE INVENTION

The present invention relates to a novel class of synthetic biocompatible cationic lipids and cationic polymers that are useful for polynucleotide packaging and delivery. In particular, the present invention describes the use of a combination of primary and secondary amines separated by, for example, ethylene hydrocarbons (i.e., multi-valent cationic groups, such as pentaethylenehexamine (PEHA)), to derivatize suitable lipid groups, e.g., phospholipid, cholesterol, etc. Consequently, an embodiment of the present invention is the novel compound triaminocholesterol (TAC) which comprises a PEHA derivative (diethylene triamine) covalently linked to cholesterol.

Additionally, the multivalent cationic groups may be assembled into cationic polymers. The cationic polymers of the present invention are comprised of substantially biocompatible cationic monomers that are interconnected by a biocompatible or substantially biocompatible linking groups. Preferably, the chemical linkages used to construct the cationic polymers of the present invention are hydrolyzable under physiological conditions or, more preferably, are biodegradable.

Typically, the cationic moieties are linked by biocompatible covalent bonds such as a disulfide bonds, hydrolyzable bonds, pH sensitive bonds, or any combination thereof.

Further embodiments of the present invention include stable polynucleotide delivery vehicles comprising the novel cationic lipids and/or cationic polymers, and methods for producing and using the same.

An additional embodiment of the present invention is a compound having the general formula:

(R₁,R₂)N—C(O)O—Y

wherein R₁ and R₂ are drawn from the group consisting of H, C₁-C₆ alkyls, alkenyls, or alkynyls, or mono or multivalent cationic amine groups (e.g., spermine, spermidine, pentaethylenehexamine (PEHA), diethylene triamine, pentamethylenehexamine, pentapropylenehexamine, etc.), and Y is a cholesterol or cholesterol derivative.

An additional embodiment of the subject invention involves biocompatible cationic polymers having the general formula:

. . . (R—X—R)n . . .

wherein R is a cationic group capable of binding nucleic acid, and X is a biocompatible, biodegradable or otherwise labile covalent cross-linker molecules and n is number between about ten and up to about ten thousand. A preferred average molecular weight for polymer preparations will typically be between about 40,000 daltons and about 1,000,000, more typically between about 60,000 daltons and about 250,000 daltons, preferably between about 80;000 daltons and about 150,000 daltons, and more preferably between about 90,000 daltons and about 110,000 daltons. Alternatively R may comprise any one of a group of cations that are used to make a heteropolymer.

Additionally, heteropolymeric cations are contemplated which have the general structure:

( . . . (X—Rₓ—X—R_{y}—X) . . . )

wherein Rₓ is a given cation that is capable of interacting with nucleic acid; R_{y} is any of a number of cations other then Rₓ that is also capable of interacting with nucleic acid; and X is a biocompatible cross-linker molecule.

Another embodiment of the present invention includes a novel process for making polynucleotide delivery vehicles comprising the steps of complexing the polynucleotide and cationic polymer and/or cationic lipid in buffer that maintains DNA as a B-form helix (e.g., an aqueous alcohol solution), and removing the buffer by evaporation. After reconstitution of the dried polynucleotide-cationic lipid/cationic polymer complex with aqueous solution, stable polynucleotide delivery vehicles are produce.

Another embodiment of the present invention contemplates the use of polynucleotide delivery vehicles comprising cationic lipids and/or polymers to deliver a polynucleotide, or polynucleotides, of interest to a cell. Accordingly, the described cationic polymers and cationic lipids may be used to provide a therapeutic benefit to an individual.

### 4.0. DESCRIPTION OF THE FIGURES

FIG. 1 shows how and where representative cationic groups may be polymerized using an appropriate dicarboxylic acid linker molecule.

FIG. 2 provides additional examples of several alternative cationic groups and linking agents that may be used to produce cationic polymers.

FIG. 3 shows a schematic synthesis scheme for the production of a novel cationic phospholipid.

FIG. 4 shows a schematic synthesis scheme for triaminocholesterol.

FIG. 5 shows in vivo expression data obtained using the PDVs produced by the ethanol evaporation method.

### 5.0. DETAILED DESCRIPTION OF THE INVENTION

The biocompatible cationic polymers or cationic lipids f the present invention may be contacted (ion paired) with a polynucleotide, or polynucleotides, of interest such that the positive charge of the cationic groups electrostatically interacts with the negatively charged polynucleotide. The electrostatic interaction between the cationic moiety and the polynucleotide presumably reduces charge repulsion in the polynucleotide and allows the polynucleotide to be condensed into a more compact configuration (as seen by gel-shift assays, etc.).

The cationic component used in the presently described cationic lipids and polymers may be monovalent, divalent, multivalent, or preferably polyvalent (i.e., polycationic). Examples of monovalent cations capable of associating with DNA include primary amines, including, but not limited to methylamine, ethylamine, etc.), and multivalent amines such as, but not limited to, spermine, spermidine, pentaethylenehexamine, diethylene triamine, pentamethylenehexamine, pentapropylenehexamine. The cationic component is preferably biocompatible or biotolerable. The cationic component may comprise any of a variety of chemical groups that retain a positive charge between pH 5 through pH 8 including, but not limited to, amino groups (or oligo or poly amines), e.g., spermine, spermidine, pentaethylehehexamine (PEHA), diethylene triamine, pentamethylenehexamine, pentapropylenehexamine, etc.), amide groups, amidine groups, positively charged amino acids (e.g., lysine, arginine, and histidine), imidazole groups, guanidinium groups, or mixtures and derivatives thereof.

The cationic component will generally be combined with the polynucleotide at a cation/phosphate ratio that has been optimized for a given application. Usually, the cation/phosphate ratio will be between about 0.1 and about 20, often between about 5 and about 17, and preferably between about 6 and about 15. The charge ratio will vary accordingly depending on the number of positively charged groups contained on the cation, and the size of the polynucleotide. For example, where DOSPA is concerned, a DOSPA/DNA nucleotide ratio of about 0.6 is suitable.

Because of the inherent toxicity of presently available cationic lipids, i.e., DOSPA, such lipids are generally not preferred for in vivo gene delivery. Consequently, cationic lipids having reduced toxicity are preferred facilitators of nonviral delivery of polynucleotides in vivo. Accordingly, additional embodiments of the present invention are novel cationic lipids produced by reacting multivalent cationic (amino) groups with, for example, cholesterol or DOPE.

Where the cationic group is attached to a phospholipid, a preferred embodiment of the present invention is a compound having the formula:

R—N—(CH)ₙ-Phospholipid

wherein the phospholipid is attached by a phosphodiester linkage; n is about 1 to about 6, and R is drawn from the group consisting of:

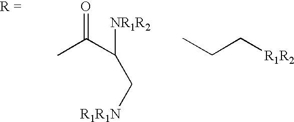

wherein R₁ and R₂ are drawn from the group consisting of H, methyl, ethyl, —C₁-C₄ alkyls, alkenyls, or alkynyls, —(CH₂)ₙNH₂, —(CH₂)ₙNH(CH₂)ₙNH₂, —(CH₂)ₙN(CH₃)₃_{⁺}, —(CH₂)ₙNH(CH₂)ₙN(CH₃)₃_{⁺}, and —(CH₂)ₙNH(CH₂)ₙNH(CH₂)ₙNH₂, n=1 to 6.

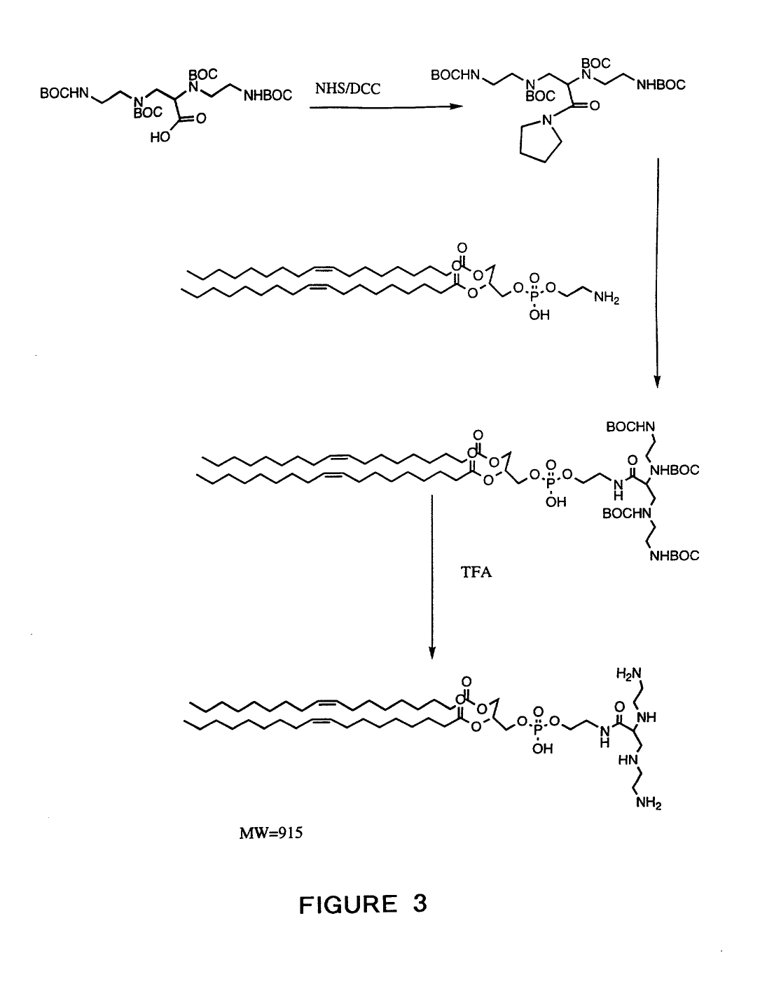

A particular example of one such cationic lipid includes the cationic phospholipid produced essentially as shown in FIG. 3.

Another embodiment of the present invention is a derivatized cholesterol compound having the structure:

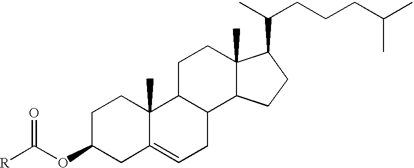

wherein R is drawn from the group consisting of:

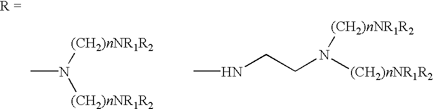

wherein R₁ and R₂ are drawn from the group consisting of H, methyl, ethyl, —C₁-C₄ alkyl, alkenyl, or alkynyls, —(CH₂)ₙNH₂, —(CH₂)ₙNH(CH₂)ₙNH₂, —(CH₂)ₙN(CH₃)₃_{⁺}, —(CH₂)ₙNH(CH₂)ₙN(CH₃)₃_{⁺}, and —(CH₂)ₙNH(CH₂)ₙNH(CH₂)ₙNH₂, n=1 to 6. Additionally, one skilled in the art would recognize that a wide variety of cholesterol derivatives, and related compounds, could be similarly derivatized with suitable cationic groups.

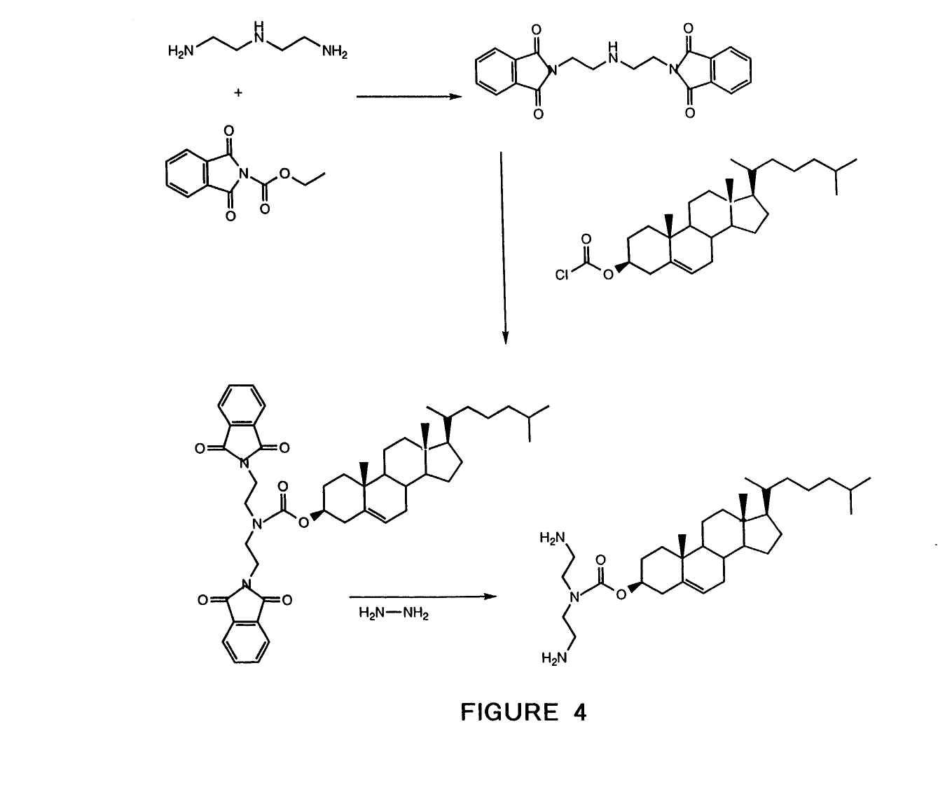

A particular example of one such cationic lipid is the molecule triaminocholesterol (TAC). TAC was constructed by reacting a diethylene triamine derivative of PEHA to a suitably treated cholesterol derivative essentially as shown in FIG. 4.

In addition to cationic lipids, similar chemistry may be used to produce cationic polymers. The cross-linking agents used to prepare the presently described polymers are preferably biocompatible or biotolerable, and will generally comprise at least two chemical groups (i.e., the cross-linkers are bifunctional) that are each capable of forming a bond with a suitable chemical group on the cation. The linker groups may be homobifunctional (same chemical groups) or heterobifunctional (different chemical groups). Preferably, the chemical linkage formed between the linking group and the cationic moiety will be hydrolyzable under physiological conditions (i.e., pH labile, or otherwise subject to breakage in the target cell). Additionally, the cross-linking agent may comprise a bond that is hydrolyzable under physiological conditions in between the linking groups.

Optionally, the cross-linking agent may be combined with an additionally cross-linking agent that a allows for the formation of branched polymers. By varying the ratio of the branching linking molecules to polymerizing cross-linker, cationic polymers are produced with a variety of chemical characteristics.

Typically, the cationic and linker components of the claimed cationic polymers are described in, or may be obtained from any of a variety of sources including, but not limited to, the 1995 edition of the Merck Index, Budavari, et al., eds., Merck and Company, Inc, Rahway, N.J., the 1995 SIGMA chemical company catalogue, St. Louis, Mo., the 1995 Aldrich Biochemicals Catalogue, or the 1995 Ofatlz and Bauer catalogue.

The cationic group may preferably be attached to the cross-linker by an amide, ester, or phosphodiester linkage which renders the linker separable from the cationic group under physiological conditions or by the action of natural enzymes such as glycosylases, proteases, lipases or phospholipases, and the like. Such a linkage represents an improvement over the currently available synthetic cationic lipids which are inherently toxic. An additional feature of the presently described polymerization reaction is that, preferably, practically useful cationic polymers may be formed without strictly requiring the employment of protecting groups, or elaborate deprotecting schemes.

One embodiment of the present invention is the use of linker molecules that are at least multicarboxylic acid derivatives of carbohydrates to form cationic polymers. Typically, the molecules will be at least dicarboxylic derivatives of carbohydrates (i.e., mono, di, or polysaccharide molecules), and will cross-link the cationic moieties by amide linkages. Alternatively, polymeric carbohydrates (i.e., similar to murein) are contemplated that are derivatized with amines, polyamines, or cationic amino acids in place of N-acetyl groups, or muramic acid.

In addition, where the cationic group is a polyamine, virtually any compound comprising dicarboxylic acid groups may act as a suitable linker molecule. Preferably, the linkers will be soluble under aqueous conditions, and the carboxylic acid groups will generally have a least one to three carbon atoms interspersed between the groups. In order to increase the solubility of the linker, it may be preferable to employ dicarboxylic acids that incorporate additional groups that increase hydrophilicity, while not substantially interfering with the polymerization reaction (i.e., hydroxyl groups or poly ethers).

Additional linker molecules include the general type, or molecules employing a similar chemical strategy, described in U.S. Pat. No. 4,833,230, herein incorporated by reference.

Additionally contemplated are linkers that form acid labile bonds upon reaction with amino groups. Such pH labile bonds comprise working exemplifications of the claimed pH sensitive/labile covalent linker moieties (which may also include ester linkages).

For the purposes of the present invention, the term “biodegradable cationic polymer” shall refer to the fact that upon entering into the cell the cationic polymer is converted to components (and metabolizable byproducts thereof) that are generally capable of participating in the catabolic or metabolic processes of the cell, or are excreted by the cell and voided. The term “biocompatible” shall mean that the compound does not display significant toxicity or adverse immunological effects at the contemplated dosages. The term “biotolerable” shall mean that an item or compound may be used to treat animals or animal cells with manageable side-effects or toxicity effects. The term “pH sensitive” shall mean that at least one covalent bond in the molecule may be broken by a change in pH that generally approximates that which occurs after endosomal fusion. The term substantially toxic shall mean that, at therapeutic dosages, a given agent produces harmful consequences which, on balance, clearly outweigh the contemplated therapeutic benefits of the agent.

Another method of polymerizing spermine, PEHA, or other cations, using a biodegradable linkage involves using dipeptide linkers which are susceptible to proteolytic cleavage by lysosomal proteases, including, but not limited to, thioproteases or cathepsins.

Additional embodiments of the present invention are novel methods of using the above-described cationic polymers and cationic lipids to deliver polynucleotides to cells in vitro or in vivo. When used for gene delivery, the cationic polymers and cationic lipids may be used in conjunction with conventional lipids, or currently available cationic lipid conjugates (e.g., Lipofectin, Lipofectamine, and the like). Preferably, the gene delivery is conducted using a method that is substantially nontoxic to the cells or patient.

The presently described cationic polymers will generally form structures in aqueous solution that are characteristic of a given polymer. In general, the polymers form a relatively compact structure in water, swell in the presence of added salt, and form an intermediate sized structure when polynucleotide is added. The changes in the physical size and density of the molecule before and after polynucleotide association allow one to follow the progress of polynucleotide association, and facilitate the isolation of the desired product.

Polynucleotide delivery vehicles (PDVs) comprising the disclosed cationic lipids or cationic polymers, or a mixture thereof, generally incorporate the polynucleotide to be delivered as a structural component of the PDV. As such, the structure of the polynucleotide contributes to the structural characteristics of the PDV. Typically, where the polynucleotide is in the form of a plasmid, the DNA will generally comprise either super-coiled or relaxed circles, or a mixture thereof. To the extent that a specific form may be preferred for a given application, enzymes such as DNA gyrase, ligase, and topoisomerase may be used to alter the structure of the plasmid as deemed necessary. Where linear polynucleotides are preferred, plasmids may be linearized, and optionally concatamerized, prior to complex formation.

Single- and double-stranded polynucleotides might also be “prepackaged” prior to complex formation by the addition of suitable polynucleotide binding proteins such as viral proteins, single-stranded binding protein, histone proteins and the like.

Polynucleotides of interest that may be delivered using the claimed polynucleotide delivery vehicles include, but are not limited to, DNA, RNA, polynucleotides associated with procaryotic and eucaryotic viral particles (e.g., retroviral core particles, bacteriophage particles, adenovirus particles, adenoassociated virus core particles, and the like), protein/DNA complexes, i.e., proteins for integration, endosome disruption, to facilitate gene transfer and expression, etc.; RNA/DNA complexes, and any and all derivatives and variations of the above. Where a DNA molecule is to be delivered, it will typically comprise a gene of interest, or portion thereof, which is flanked by regulatory sequences which are spatially organized to optimize the expression of the DNA of interest.

Preferably, the polynucleotide to be delivered using the presently described PDVs will be substantially pure (i.e., substantially free of contaminating proteins, lipid, polysaccharide, lipopolysaccharide, and nucleic acid). For example, where plasmid DNA is used, the preparations will generally be prepared by a process comprising phenol, or phenol:chloroform, extraction, and isopycnic centrifugation (using CsCl, and the like), or functional equivalents thereof. Preferably, the DNA preparations will also be treated with RNase, and subject to multiple rounds of extraction, and at least two rounds of ultracentrifugation (or any other means of producing DNA at least as pure). Typically, a substantially pure preparation of nucleic acid is a preparation in which at least about eighty percent, generally at least about ninety percent, and preferably at least about ninety five percent of the total nucleic acid is comprised of the desired nucleic acid.

Genes of interest are typically inserted into any of a wide range of expression vectors which are subsequently delivered using the presently disclosed methods and materials. Suitable vectors which may be delivered using the presently disclosed methods and compositions include, but are not limited to, herpes simplex virus vectors, adenovirus vectors, adeno-associated virus vectors, retroviral vectors, pseudorabies virus, alpha-herpes virus vectors, and the like. A thorough review of viral vectors, particularly viral vectors suitable for modifying nonreplicating cells, and how to use such vectors in conjunction with the expression of polynucleotides of interest can be found in the book Viral Vectors: Gene Therapy and Neuroscience Applications Ed. Caplitt and Loewy, Academic Press, San-Diego (1995). It is contemplated that the subject methods and compositions may be used to directly deliver vector nucleic acid, or, where applicable, viral or subviral particles encoding or containing the nucleic acid of interest.

As used herein, the term “expression” refers to the transcription of the DNA of interest, and the splicing, processing, stability, and, optionally, translation of the corresponding mRNA transcript. Depending on the structure of the DNA molecule delivered, expression may be transient or continuous.

Any number of transcriptional promoters and enhancers may be used in the DNA of interest, including, but not limited to, the herpes simplex thymidine kinase promoter, cytomegalovirus promoter/enhancer, SV40 promoters, and retroviral long terminal repeat (LTR) promoter/enhancers, and the like, as well as any permutations and variations thereof, which may be produced using well established molecular biology techniques (see generally, Sambrook et al. (1989) Molecular Cloning Vols. I-III, Cold Spring Harbor Laboratory Press, Cold Spring Harbor, N.Y., and Current Protocols in Molecular Biology (1989) John Wiley & Sons, all Vols. and periodic updates thereof, herein incorporated by reference).

Promoter/enhancer regions may also be selected to provide tissue-specific expression. Typically, where translation is desired, the genes of interest will also be engineered to comprise a suitable 3′ polyadenylation sequence (if necessary).

DNAs of particular interest include, but are not limited to, sequences encoding a variety of proteins, cytokines and growth factors, (such as, G-CSF, GM-CSF, nerve growth factor (NGF), ciliary neurotropic factor (CNTF), brain-derived neurotropic factor (BDNF), interleukins 1-2 and 4-14, tumor necrosis factor-α (TNF-α), α or γ interferons, erythropoietin, and the like), the cystic fibrosis transmembrane conductance regulator (CFTR), tyrosine hydroxylase (TH), D-amino acid decarboxylase, GTP cyclohydrolase, leptin, leptin receptor, factors VIII and IX, tissue plasminogen activator (tPA).

The term “biologically active material” includes, in particular, pharmaceutically active proteinaceous materials, and pharmaceutically active organic molecules.

Additionally, antisense, antigene, or aptameric oligonucleotides may be delivered using the presently described PDVs. Ribozymes, RNA-DNA hybrids, polynucleotide peptide bonded oligos (PNAs), circular or linear RNA, circular single-stranded DNA.

RNAs of interest include self-replicating RNAs, mRNA transcripts corresponding to any of the above genes which may be directly translated in the cytoplasm, or catalytic RNAs, e.g. “hammerheads” hairpins, hepatitis delta virus, group I introns which may specifically target and/or cleave specific RNA sequences in vivo. Of particular interest for targeting by catalytic RNAs are RNA viruses, as well as both cellular and viral transcripts.

Alternatively, antisense forms of RNA, DNA, or a mixture of both may be delivered to cells to inhibit the expression of a particular gene of interest in the cell or to correct point, or other (nonsense or missense, etc.) mutations.

An additional embodiment of the present invention contemplates the delivery of oligomeric nucleotides which have been incorporated into the PDVs in conjunction with larger polynucleotides. Such “carrier” polynucleotides may be single-stranded (linear or circular), or substantially double-stranded, and may additionally comprise one or more regions which are substantially homologous or complementary to the oligomeric nucleotides to be delivered.

When desired, the DNA of interest may further incorporate a suicide signal that allows for the controlled extermination of cells harboring and expressing the DNA of interest previously delivered by the delivery vehicle. For instance, the thymidine kinase (tk) gene may be incorporated into the delivered DNA which would allow the practitioner to subsequently kill cells expressing the tk gene by administering the correct amounts of acyclovir, gangcyclovir, or the conceptual or functional equivalents thereof.

The presently described methods for producing polynucleotide delivery vehicles require that the polynucleotide(s) of interest be contacted with the amphipathic cationic lipid conjugates such that ion pairing between the cationic moiety and the polynucleotide allows for complex formation. The condensed cationic polymer/lipid-polynucleotide complex may subsequently serve as a scaffold, or nucleus, for the assembly of polynucleotide delivery vehicles (PDVs). Alternatively, the cationic polymer/lipid-polynucleotide complex may be used directly.

Given that ion pairing plays a role in the formation of the polynucleotide delivery vehicle, the pH during complex formation may be varied to optimize or stabilize the interaction of the specific components. For instance, where non-pH sensitive cationic polymers are used, a pH as low as about 4 may be preferred to complex a given polynucleotide (e.g., RNA) or other chemical agent which may be coincorporated with the polynucleotide. Additionally, where the polynucleotide (e.g., DNA) is not substantially sensitive to base hydrolysis, circumstances may dictate that a pH of up to about 10 be used during complex formation. Generally, a pH within the range of about 5 to about 9 will be maintained during complex formation and transfection.

Similarly, the concentration of salt (e.g, NaCl, KCl, MgCl₂, etc.) may be varied to optimize complex formation, or to enhance the efficiency of gene delivery and expression. Additionally, factors such as the temperature at which the cationic lipids or cationic polymers are complexed may be varied to optimize the structural and functional attributes of the resulting PDVs. Additionally, the osmolarity of solution in which the complexes are formed may be altered by adjusting salt concentration.

Given that moderate concentrations of salt may impede complex formation, one may also adjust osmolarity by adding or substituting suitable excipients such as, but not limited to, glucose, sucrose, lactose, fructose, trehalose, maltose, mannose, and the like.

Typically, many cationic condensing agents (e.g., spermine, PEHA, or spermidine) will precipitate polynucleotide. However, the carefully controlled addition of condensing cationic polymer to the polynucleotide (using an infusion pump or the like) allows for relatively high concentrations of polynucleotide (e.g., about 0.5 mg/ml) to be complexed with the condensing agent. As such, carefully controlled addition of the polynucleotide to the cationic condensing agent allows for relatively high concentrations of polynucleotide to be complexed by the cationic condensing agent.

Where cationic or neutral lipids are to be used in conjunction with the cationic polymers, the lipid is generally dissolved or solubilized in a suitable detergent in order to form lipid micelles. Detergents suitable for dissolving lipids include, but are not limited to cholate, deoxycholate, lauroyl sarcosine, octanoyl sucrose, CHAPS (3-[(3-cholamidopropyl)-di-methylamine]-2-hydroxyl-1-propane), novel-β-D-glucopyranoside, Lauryl dimethylamine oxide, octylglucoside, and the like. Preferably, the detergent will be nonionic and possess a high critical micelle concentration (CMC). When the polynucleotide and cationic polymer are added to the micellized amphipathic cationic lipid conjugate, ion pairing occurs and the polynucleotide condenses as a complex with the cationic components.

After initial complex formation, slow removal of the detergent (i.e., by extensive dialysis) allows for the assembly and formation of lipid associated polynucleotide delivery vehicles. While slow dialysis remains the preferred method of detergent removal, one may expedite detergent removal by increasing the relative amount of dialysis buffer or by adding a reagent to the buffer which binds and removes the detergent from the dialysate buffer solution.

Alternatively, the polynucleotide and cationic polymer may be dissolved in a solution containing a suitable cation prior to the addition of lipid and detergent. After the detergent is added, it is removed by dialysis in the presence of cation, and subsequently the cation may removed by dialysis. Suitable cations include any element carrying a positive charge. The cation may be monovalent, divalent, or multivalent. Typical examples of suitable elemental cations include, but are not limited to manganese, magnesium, sodium, calcium, rubidium, zinc, molybdenum, nickel, iron and the like. Generally, the elemental cation will be added in an amount sufficient to prevent aggregate formation during complexation of the lipid and the polynucleotide, and up to a concentration of about the maximum solubility of a given cationic compound. Preferably, the concentration of sodium, (e.g., sodium chloride) will be between about 0.1 molar and about 5 molar, the concentration of magnesium (e.g., magnesium chloride) will be between about 0.05 molar and about 5 molar; and the concentration of manganese (e.g., manganese chloride) will be between about 0.1 molar and about 4 molar.

Additionally, one of ordinary skill will appreciate that the type and concentration of cation may have to be adjusted depending on the characteristics of the cationic polymer used to assemble the PDVs.

Where the polynucleotide, or oligonucleotide, is to be complexed with cation during the assembly of PDVs, the cationic polymer or cationic lipid (molecular cations), and/or detergent may be added prior to, concurrently with, or subsequent to, the addition of cation. Generally, the cationic polymer and cationic lipid will be added to the poly, or oligo, nucleotide at a net molecular cation-to-polynucleotide phosphate ratio of between about 0.1:1 and about 16:1, preferably between about 0.5:1 and about 7:1, more preferably between about 0.7:1 and about 2:1, and specifically about 1:1. The above ratios are provided for exemplification and not limitation, and may be modified depending on the characteristics of the molecular cations used to assemble the PDVs. Also, the optional ratio will be dependent upon the DNA concentration.

After the cation, poly or oligonucleotide, cationic polymer, cationic (or other) lipid, and detergent are present in the milieu, the detergent will preferably be removed by dialysis in cation comprising buffer. After the detergent is removed, the cation may subsequently be substantially removed by dialysis, or a functional equivalent. Preferably, dialysis will generally be performed at a temperature of between about 4° C. and about 30° C., and will result in a final cation concentration that is not detrimental to the intended use of the PDV. For instance, the elemental cation may be substantially removed by, for example, dialysis with a buffered solution that is suitable for parenteral administration.

After the substantial removal of the cation, the resulting PDVs generally remain stable (i.e., retain transduction activity) for at least two weeks when stored at about 4° C., or may be lyophilized and stored indefinitely.

Because the presently described cationic polymers are preferably biocompatible, PDVs comprising the cationic polymers will bear reduced toxicity. For the purposes of the present disclosure, reduced toxicity shall mean that PDVs comprising at least about 10 μg of DNA may be injected into an animal without the animal suffered grave toxicity effects.

By increasing the concentration of elemental cation used to precondense the polynucleotides, one may increase the concentration of DNA used to assemble the PDVs by a corresponding amount (i.e., 2 molar MnCl₂ may allow for PDV formation at a concentration of about 1 mg/ml of DNA). Given the relatively high solubility of the applicable cations (i.e., NaCl saturates at about 5.5 molar), it is clear that the present methods enables the formation of PDVs at a concentration of at least about 10 mg/ml of DNA (or other polynucleotide). Accordingly, another embodiment of the present invention are preparations of PDVs that have been formulated as described above and comprise a concentration of DNA (or other nucleotide) of generally between about 0.05 mg/ml and about 10 mg/ml, preferably between about 0.25 mg/ml and about 10 mg/ml, more preferably between about 0.5 mg/ml and about 1.5 mg/ml, and specifically between about 0.8 mg/ml and 1.2 mg/ml. Accordingly, another embodiment of the present invention are PDV compositions comprising high concentrations of nucleic acid (i.e. >0.25 mg/ml nucleic acid).

Formulating PDVs using the detergent dialysis method described above typically generates particles that are greater than 200 nm in mean diameter. Where smaller particles may be preferred, an alternative method for formulating PDVs involves forming the cationic lipid-polynucleotide complex in a mixed aqueous solution that has been formulated to maintain the polynucleotide in a structural conformation that is conducive to binding by cationic lipids or cationic polymers. Examples of such solutions include mixed water/alcohol (e.g., methanol, ethanol, isopropanol, butanol, and isomers and mixtures thereof, etc.) solutions. Preferably, such complexation buffers also contain a concentration of dissolved sugar and/or salt.

Generally, the concentration of alcohol (e.g., ethanol) present during complex formation shall range from between about 10 percent up to about 80 percent, typically between about 20 percent and about 50 percent, more typically between about 30 percent and about 45 percent, preferably between about 37 percent and about 43 percent, and more preferably about 40 percent.

The amount of sugar (dextrose, sucrose, etc., see list provided above) that may be present during complex formation shall generally vary from between about 2 percent and about 15 percent, preferably between about 3 percent and about 8 percent, and more preferably about 5 percent.

Alternatively, the osmolarity of the solution may also be adjusted by a mixture of salt and sugar. One skilled in the art would clearly know how to appropriately vary the concentration of salt and sugar to optimize the efficiency of gene delivery. Typical concentrations of salt and sugar that may serve as a starting point for further optimization are 250 mM (glucose) and 25 mM salt (NaCl).

An additional feature of complex formation is temperature regulation. Typically, cationic lipids or polymers are complexed with polynucleotide at a temperature between about 4° C. and about 65° C., more typically between about 10° C. and about 42° C., preferably between about 15° C. and about 37° C., and more preferably at about room temperature. In many instances, precise regulation of temperature is important to minimizing product variability.

After the solution is removed from the complex by, for example, evaporation, the dry complex remains stable and may be stored indefinitely. After reconstitution, the size of the complex may be further adjusted by established means such as extrusion, homogenization, sonication, and the like.

Because polynucleotide delivery vehicles comprising the described cationic polymers or cationic lipids form particles of discreet size, targeting agents may be stably incorporated into the vehicles to direct the vehicles to specific cells and/or tissues. Accordingly, any of a variety of targeting agents may be also be incorporated into the delivery vehicles.

For the purposes of the present disclosure, the term targeting agent shall refer to any and all ligands or ligand receptors which may be incorporated into the delivery vehicles. Such ligands may include, but are not limited to, antibodies such as IgM, IgG, IgA, IgD, and the like, or any portions or subsets thereof, cell factors, cell surface receptors, MHC or HLA markers, viral envelope proteins, peptides or small organic ligands, derivatives thereof, and the like.

The targeting ligand may be derivatized to an appropriate portion of the cationic polymer prior to the formation of the polynucleotide delivery vehicle. For example, the targeting agent (e.g., immunoglobulin) may be N-linked to a free carboxyl group of the polar region of a branched cross-linking molecule, by first derivatizing a leaving group to the carboxyl group using N-hydroxysuccinimide (NHS) and 1-ethyl-3-(3-dimethylaminopropyl)carbodiimide (EDAC), or the methiodide thereof, (EDC methiodide) and a free amino group on the targeting molecule. Alternatively, targeting agents may be disulfide linked to a properly conditioned linking agent or cation (using thioacetic acid, hydroxylamine, and EDTA).

Where the PDVs comprise lipids, succinimidyl acetylthioacetate may be used in conjunction with a fatty acid (e.g., dioleylphosphatidyl-ethanolamine, DOPE) to form a DOPE-thioacetate (ATA) which may be treated with hydroxylamine to generate the reduced molecule (DOPE-acetyl-SH). A free amino group on the targeting agent is reacted with succinimidyl maleimidophenyl butyrate (SMPB) to produce a target which is linked to maleimidophenylbutyrate (MPB) by a peptide bond. The derivatized fatty acid is subsequently combined with the target-MPB complex to produce a targeting agent which has been cross-linked to a fatty acid.

Additionally, the targeting agent may be linked to the lipid by a biodegradable linkage as discussed above (peptide or dipeptide linkers, pH hydrolyzable linkers, etc.).

Alternatively, the targeting agent may also act as a bridge between the PDVs and the “targeted” cells or tissues. For instance, where the targeting agent simply associates with the complex, the agent may be added to the complex well after complex formation or isolation. To the extent that the targeting agent is also capable of recognizing, or being recognized by, molecules on the cell surface, it may act as a bridge molecule which effectively places the complex in intimate contact with the cell surface.

Particularly where hepatocytes are the preferred target of PDV-directed transfection, molecules such as fetuin may prove useful. Hepatocytes contain a galactose receptor. After treatment with neuraminidase, fetuin is converted to asialofetuin which displays a number of galactose residues on its surface. Moreover, both fetuin and asialofetuin are known to associate with the DNA complexes comprising cationic lipids.

As a molecule rich in acidic amino acids (aspartic acid and glutamate) asialofetuin (ASF) presumably associates the cationic groups of DNA/cation complexes. Consequently, asialofetuin-associated complexes are targeted to hepatocytes by virtue of the exposed galactose residues on the protein.

The observation that asialofetuin associates with DNA/cation complexes also has far reaching potential. For instance, asialofetuin may be derivatized with any of a wide number to targeting ligands using any of a number of conventional chemical methods. For instance, periodate may be used to convert at least a portion of the hydroxyl groups on galactose to aldehydes, the aldehydes react with primary amino groups to form Schiff bases, which may be subsequently be reduced with lithium aluminum hydride (to add a targeting ligand). Alternatively, the aldehydes may be reacted with hydrazide to attach heterobifunctional cross-linking reagents (which has been to suitable targeting ligands). Either of the above strategies are simply illustrative of the many possible ways asialofetuin may be derivatized with practically any targeting ligand, and should not be construed as limiting the invention in any way.

After ligand association, the derivatized asialofetuin may be associated with the DNA/cation complex as described above. Virtually any ligand can be attached to asialofetuin, and virtually any DNA can be packaged into the stable complex. Thus, by carefully matching a properly derivatized asialofetuin with the appropriate DNA/cation complex, virtually any cell may be targeted to express virtually any gene.

Moreover, asialofetuin, or functional equivalents thereof (vis-a-vis binding) may be N-linked to the cationic polymer and directly incorporated into PDVs.

Additionally, it is likely that other proteins will be identified or developed that are also capable of associating with PDVs. Like asialofetuin, proteins that associate with the PDVs may be suitably derivatized with a targeting ligand and used to direct PDVs to specific cells and tissues. In this manner, any of a variety of cells such as endothelial cells, line cells, epithelial cells, islets, neurons or neural tissue, mesothelial cells, osteocytes, chondrocytes, hematopoietic cells, immune cells, cells of the major glands or organs (e.g., lung, heart, stomach, pancreas, kidney, skin, etc.), exocrine and/or endocrine cells, and the like, may be targeted for gene delivery.

Of particular interest are proteins encoding various cell surface markers and receptors. A brief list that is exemplary of such proteins includes: CD1(a-c), CD4, CD8-11(a-c), CD15, CDw17, CD18, CD21-25, CD27, CD30-45(R(O, A, and B)), CD46-48, CDw49(b,d,f), CDw50, CD51, CD53-54, CDw60, CD61-64, CDw65, CD66-69, CDw70, CD71, CD73-74, CDw75, CD76-77, LAMP-1 and LAMP-2, and the T-cell receptor, integrin receptors, endoglin for proliferative endothelium, or antibodies against the same.

For the purposes of the present disclosure, polynucleotide delivery vehicles comprising the described cationic polymers and cationic lipids generally retain transfection efficiencies of at least about twenty (20) percent of the polynucleotide transfection efficiency of freshly prepared product after storage for forty-eight (48) hours, and preferably retain at least about thirty-five (35) percent transfection efficiency after 48 hours, and in a particularly preferred embodiment will retain at least about fifty (50) percent transfection efficiency after 48 hours.

Alternatively, the presently described PDVs remain size-stable and generally retain a discrete size range of between about 50 and about 1,000 nm, preferably between about 75 and about 600 nm, and preferably between about 100 and about 450 nm average particle size (as per a Gaussian distribution) after being held in the liquid state for at least 48 hours. Generally, PDVs formed by ethanol evaporation are smaller (mean diameter less than about 150 nm) than PDVs formed by detergent dialysis (mean diameter greater than about 200 nm).

Where stability in serum is concerned, the presently described PDVs are preferably serum stable in that they are generally at least about twice as stable than, and preferably at least about one order of magnitude more stable than liposomal formulations produced using the methods/synthetic cationic lipids taught by the prior art when exposed to serum concentrations of up to about fifteen (15) percent.

The stability of the presently described PDVs may be augmented by the appropriate storage conditions. For example, the PDVs may be frozen and stored indefinitely. After rapid or slow (at about 4° C.) thawing, the PDVs typically retain a substantial portion, if not all, of the transfection efficiency of freshly produced samples. Moreover, the subject PDVs also retain a substantial amount (i.e., at least about 50 percent) of their original transfection efficiency after lyophilization and reconstitution.

Where one seeks to augment long-term stability by freezing or freeze-drying the PDVs, suitable excipients may be added to the PDV preparation prior to freezing. Examples of such stabilizing excipients include, mono or disaccharides (e.g., glucose, sucrose, etc.), polysaccharides, or any of a variety of well-known agents (e.g., glycerols, gums, dextrans, and the like).

PDVs may aggregate. For the purposes of this disclosure, a loose aggregate is defined as an aggregate that is easily dispersible into suspension. Optionally, such aggregation may occur after a period of frozen storage (at about −20° C. or less), followed by thawing. To the extent that aggregation is desirable, the level of aggregation may be regulated by any of a number of means in addition to adjusting temperature. For example, buffer/salt concentration may be adjusted to increase the amount of aggregation. Moreover, coprecipitants may be added which complex with the stable complexes and further increase the rate of extent of precipitation. Aggregation may also be increased by the addition of facilitating agents. For example, where a targeting agent or receptor is incorporated into the complex, a suitable lectin, ligand, or antibody may be added to cross-link the complexes and increase the rate and extent of aggregation or precipitation.

Where a targeting agent has been built into the PDVs, a suitable ligand or antibody, or mixture thereof, may be affixed to a suitable solid support, i.e., latex beads, microcarrier beads, membranes or filters, and the like, and used to selectively bind PDVs which incorporate the targeting receptor or ligand from the preparation. Thus, a method is provided for isolating the desired PDVs prior to use.

As a net result of aggregation, isolation and resuspension, PDVs may be obtained which both retain DNA transfection activity, and comprise DNA concentrations which far exceed the amounts of DNA that may normally be loaded into conventionally produced lipid/DNA complexes. Accordingly, an additional embodiment of the present invention is a method of producing PDVs that retain measurable transfection activity and comprise at least about 10 μg of nucleic acid per ml up to about 10 mg/ml.

Similarly, another embodiment of the present invention is a method of producing PDVs of substantially reduced toxicity. For the purposes of this disclosure, the terms “substantially reduced toxicity” or “substantially nontoxic” shall mean that the toxicity of an agent shall generally be reduced by at least about 25 percent relative to existing cation-derivatized polymers (i.e., DEAE-dextran, and the like), preferably by at least about 50 percent, and optimally a reduction of at least about a 100 percent will be achieved.

Toxicity may also be measured by determining the dose which is lethal to fifty percent of the test subjects. Generally, the described PDVs will have a lethal dose, or LD₅₀, twice that of nonisolated stable complex formed at similar cationic lipid/phosphate ratios, and optimally reduced toxicity vehicles will have an LD₅₀ at least about one order of magnitude greater than that of DEAE-dextran.

Additionally, any of a variety of stabilizing agents may be utilized in conjunction with the described vehicles. Although oxidation of the various components may be substantially reduced by preparing formulations in accordance with the present invention under an inert atmosphere, such as nitrogen, this is a somewhat inconvenient and expensive process and so it is often preferred to add chemical anti-oxidants. Suitable pharmaceutically acceptable antioxidants include propyl gallate, butylated hydroxyanisole, butylated hydroxytoluene, ascorbic acid or sodium ascorbate, DL- or D-alpha tocopherol and DL- or D-alpha-tocopherol acetate. The anti-oxidant, if present, may be added singly or in combination to the polynucleotide delivery vehicles either before, during, or after vehicle assembly in an amount of up to, for example, 0.1% (w/v), preferably from 0.0001 to 0.05%.

One of ordinary skill will appreciate that, from a medical practitioner's or patient's perspective, virtually any alleviation or prevention of an undesirable symptom (e.g., symptoms related to disease, sensitivity to environmental or factors, normal aging, and the like) would be desirable. Thus, for the purposes of this Application, the terms “treatment”, “therapeutic use”, or “medicinal use” used herein shall refer to any and all uses of the claimed compositions which remedy a disease state or symptoms, or otherwise prevent, hinder, retard, or reverse the progression of disease or other undesirable symptoms in any way whatsoever.

When used in the therapeutic treatment of disease, an appropriate dosage of polynucleotide delivery vehicle (PDV), or a derivative thereof, may be determined by any of several well established methodologies. For instance, animal studies are commonly used to determine the maximal tolerable dose, or MTD, of bioactive agent per kilogram weight. In general, at least one of the animal species tested is mammalian. Those skilled in the art regularly extrapolate doses for efficacy and avoiding toxicity to other species, including human. Before human studies of efficacy are undertaken, Phase I clinical studies in normal subjects help establish safe doses.

Particularly where in vivo use is contemplated, the various biochemical components of the present invention are preferably of high purity and are substantially free of potentially harmful contaminants (e.g., at least National Food (NF) grade, generally at least analytical grade, and preferably at least pharmaceutical grade). To the extent that a given compound must be synthesized prior to use, such synthesis or subsequent purification shall preferably result in a product that is substantially free of any potentially toxic agents which may have been used during the synthesis or purification procedures.

Additionally, PDVs may also be modified to enhance in vivo stability as well as any of a variety of pharmacological properties (e.g., increase in vivo half-life, further reduce toxicity, etc.) by established methods. For instance, by varying the extent of cross-linking and branching in the cationic polymer, the physiological characteristics of the PDVs may be altered. This makes is possible to construct PDVs that are capable of delivering nucleic acid to the body in a time-released manner. Such time release formulations are contemplated to facilitate the treatment of acute conditions by providing extended periods of transient gene delivery, or providing practitioners with alternative means of dosaging and delivering nucleic acid in vivo. In particular, the presently described PDVs are ideal for the packaging and delivery of nucleic acid based vaccines.

Where diagnostic, therapeutic or medicinal use of PDVs, or derivatives thereof, is contemplated, the PDV may be prepared and maintained under sterile conditions and thus avoid microbial contamination. Because of the relatively small size and inherent stability of the PDVs, compositions comprising PDVs may also be sterile filtered prior to use. In addition to the above methods of sterile preparation and filter sterilization, antimicrobial agents may also be added. Antimicrobial agents which may be used, generally in amounts of up to about 3% w/v, preferably from about 0.5 to 2.5%, of the total formulation, include, but are not limited to, methylparaben, ethylparaben, propylparaben, butylparaben, phenol, dehydroacetic acid, phenylethyl alcohol, sodium benzoate, sorbic acid, thymol, thimerosal, sodium dihydroacetate, benzyl alcohol, cresol, p-chloro-m-cresol, chlorobutanol, phenylmercuric acetate, phenylmercuric borate, phenylmercuric nitrate and benzylalkonium chloride. Preferably, anti-microbial additives will either enhance the biochemical properties of the PDVs, or will be inert with respect to PDV activity. To the extent that a given anti-microbial agent may prove deleterious to PDV activity, another agent may be substituted which effects PDV function to a lesser extent.

Compositions comprising PDVs as active components may be introduced in vivo by any of a number of established methods. For instance, the agent may be administered by inhalation; by subcutaneous (sub-q); intravenous (I.V.), intraperitoneal (I.P.), or intramuscular (I.M.) injection; rectally, as a topically applied agent (transdermal patch, ointments, creams, salves, eye drops, and the like), or directly injected into tissue such as tumors or other organs, or in or around the viscera.

Another embodiment of the subject invention involves the use of PDVs to effect gene therapy. Such gene therapy is intended to compensate for genetic deficiencies in the afflicted individual's genome and may be effected by ex vivo somatic cell gene therapy whereby host cells are removed from the body are transduced to express the deficient gene and reimplanted into the host. Alternatively, somatic cell gene therapy may be effected by directly injecting a vector bearing the desired gene into the individual, in vivo, whereby the gene will be delivered and expressed by host tissue.

Although the above polynucleotide delivery vehicles are primarily intended to provide polynucleotides to cells, a further embodiment of the present invention contemplates the packaging and delivery of any of a variety of suitable bioactive agents in addition to polynucleotides. For instance, to the extent that a bioactive agent (e.g., any protein, peptide, small organic molecule, and the like) of interest comprises a net negative charge or comprises a substantial amount of negatively charged character, it may prove useful to deliver such an agent using the presently described cationic polymers.

In addition, to the extent that a given agent of interest may associate with polynucleotide (e.g., proteins or other molecules with DNA and/or RNA binding activity), it may prove useful to deliver the agents to the body by first incorporating them into the described polynucleotide delivery vehicles.

If desired, one or more stabilizers and/or plasticizers may be added to PDV formulations for greater storage stability. Materials useful as stabilizers and/or plasticizers include simple carbohydrates including, but not limited to, glucose, galactose, sucrose, or lactose, dextrin, acacia, carboxypolymethylene and colloidal aluminum hydroxide. When stabilizers/plasticizers are added, they may be incorporated in amounts up to about 10% (w/v), preferably from about 0.5 to 6.5%, of the total preparation.

Lipid formulations (e.g., emulsions, microemulsions, liposomes, or delivery vehicles) may also significantly protect PDVs from the digestive process. As so formulated, PDVs may also prove useful for the oral administration of bioactive agents. To the extent that additional enteric protection is desired, it is possible to formulate solid or liquid formulations in accordance with the invention in an enteric-coated or otherwise protected form. In the case of liquid formulations, they can either be mixed or simply coadministered with a protectant, such as a liquid mixture of medium chain triglycerides, or they can be filled into enteric capsules (for example of soft or hard gelatin, which are themselves optionally additionally enteric coated. Alternatively, solid, or dry (i.e., desiccated or lyophilized), formulations of PDVs may be treated more flexibly. They may either be coated with enteric materials to form tablets or they can be filled into enteric capsules.

The thickness of enteric coating on tablets or capsules can be, for example, from 0.5 to 4 microns in thickness, although the precise thickness will be determined by the skilled formulator. Enteric coated granules (whose particle size may be, for example, from 0.5 to 2 mm) may themselves be coated without being compounded into a tablet for coating. Microcapsules, similarly, can be enteric coated. The enteric coating may comprise any of the enteric materials conventionally utilized in orally administrable pharmaceutical formulations. Suitable enteric coating materials are known, for example, from “Remington's Pharmaceutical Sciences”, 15th Edition, pp. 1614-1615 (1975); 2nd Edition, pp. 116-117, 371-374 (1976); and “Hagars Handbuch der Pharmazeutischen Praxie”, 4th Edition, Volume 7a (Springer Verlag 1971), pages 739 to 742 and 776 to 778.

Examples of suitable enteric coating materials include cellulose acetylphthalate, hydroxypropylmethylcellulose-phthalate (HPMC-P), benzophenyl salicylate, cellulose acetosuccinate, copolymers of styrene and maleic acid, formulated gelatin, keratin, stearic acid, myristic acid, polyethylene glycol, shellac, gluten, acrylic and methacrylic resins and copolymers of maleic acid and phthalic acid derivatives. The enteric coating material(s) may be dissolved in solvents such as dichloromethane, ethanol and water, cellulose phthalate, or polyvinyl acetate phthalate. It is preferred to utilize HPMC-P, polyethylene glycol 6000 or shellac as the enteric coating. A proprietary preparation of HPMC-P aimed at dissolution or dissipation at pH 5.5, which is encountered in the human pyrolus, is available under the trade mark HP5-5, and is particularly preferred.

The presently disclosed cationic polymers, and polynucleotide delivery vehicles produced therewith, represent a marked improvement over currently available synthetic cationic lipids vis-a-vis polynucleotide delivery to cells because the byproducts of the degradation reaction are substantially nontoxic, or inherently biocompatible. As such, the presently disclosed cationic polymers are be useful for the delivery of polynucleotides to cells in vitro as well as in vivo.

Another embodiment of the present invention is the use of the biocompatible pH sensitive or otherwise biodegradable linker portion of the cationic polymer to attach other biocompatible or groups in place of the presently disclosed cationic groups. For instance, bioactive molecules may be functionally derivatized to polymers as described above and delivered to the body in a controlled release manner. Examples of proteinaceous biological material which may be used in accordance with this invention include, but are not limited to, protein hormones such as insulin, calcitonin and growth hormone, whether from human or animals or semi- or totally synthetically prepared, erythropoietin, plasminogen activators and their precursors, such as tPA, urokinase, pro-urokinase and streptokinase, interferons including human interferon alpha, interleukins including IL-1, IL-2, IL-3, IL-4, IL-5, IL-6, IL-7 and IL-12, and blood factors including Factor VIII.

Alternatively, biocompatible anionic polymers produced using similar technology are also contemplated which will provide their unique advantages to drug packaging and delivery.

The examples below are provided to illustrate the subject invention. Given the level of skill in the art, one may be expected to modify any of the above or following disclosure to produce insubstantial differences from the specifically described features of the present invention. As such, the following examples are provided by way of illustration and are not included for the purpose of limiting the invention.

### 6.0. EXAMPLES

6.1. Methods of Making Cationic Polymers

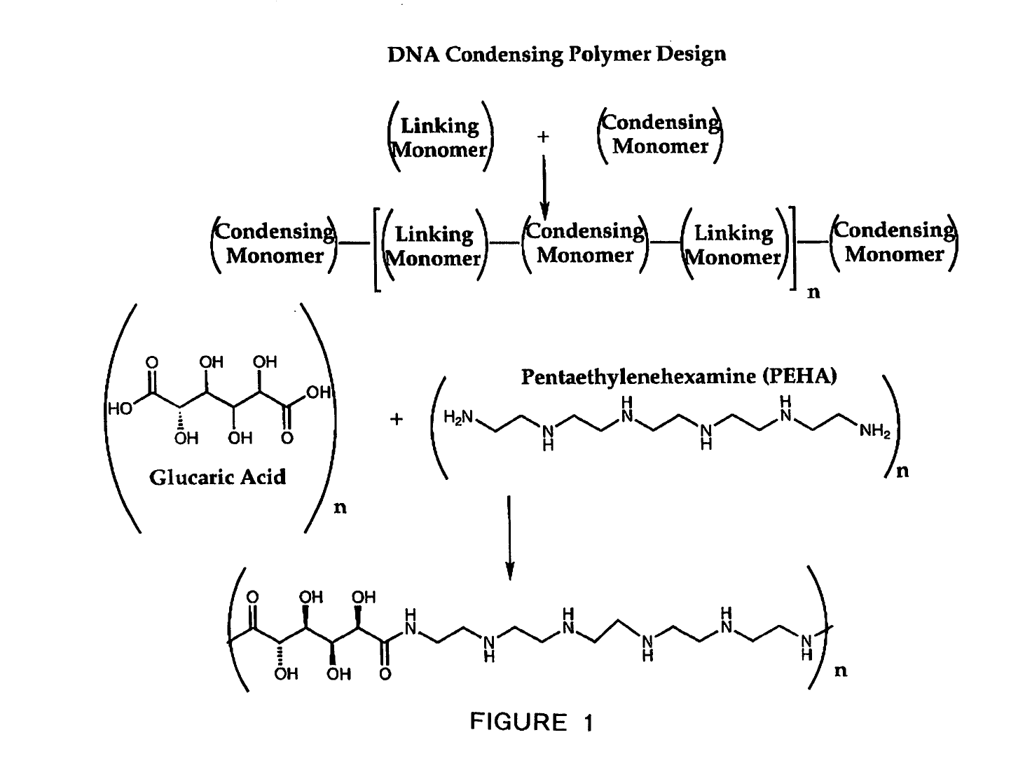

FIG. 1 provides an overview of a method for producing cationic polymers. The polycation PEHA is specifically shown but it is contemplated that similar cations comprising lesser, greater, or more highly branched amine groups are equally useful. The linking group shown in FIG. 1 is a dicarboxylic acid that cross-links the PEHA monomers by amide linkages.

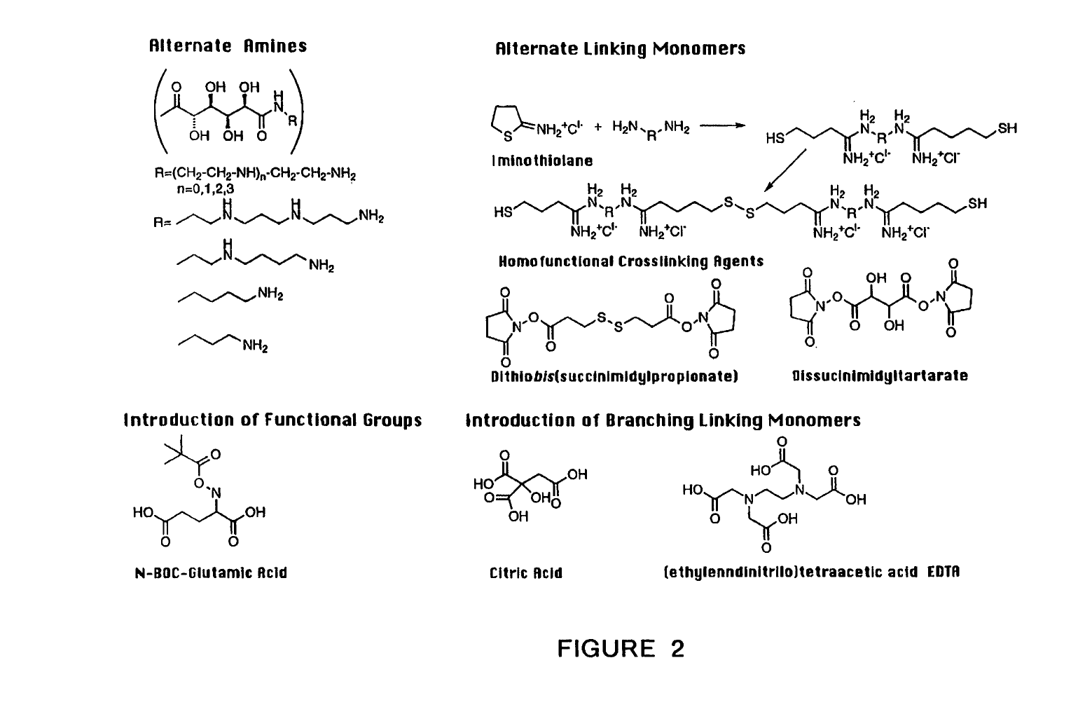

FIG. 2 provides a description of a fraction of the various other cationic groups, cross-linking agents, functional groups, and branched cross-linking agents, that may be also be used to construct cationic polymers for gene delivery. In particular, alternate biogenic amines are shown. In addition, the homofunctional cross-linking agents iminothiolane, dithiobis (succinimidylpropionate), and disuccinimidyltartarate are shown. The molecule N-BOC-glutamic acid is provided as an example of how additional functional groups may be incorporated into the cationic polymer (using carboxylic acid groups on the amino acid), and the use of citric acid and ethylenediaminetetraacetic acid (EDTA) as branched cross-linking agents is also shown.

6.1.1. Reagents

Reagent grade PEHA was obtained from Aldrich chemicals and analysis showed that the molecule, as provided, was about eighty percent full-length product mixed with a variety of shorter, and longer, synthesis products. Where in vivo use is contemplated, all reagents will be of the highest purity available, and preferably of pharmaceutical grade or better.

6.1.2. Cationic Polymer Production

PEHA is polymerized using cross-linker by slowly adding PEHA to an excess of linker (with stirring at room about room temperature). During the reaction, the resulting polymer may precipitate from solution and facilitate isolation of the product. Alternatively, the relative concentrations of the reagents may be reversed. By varying the duration of the polymerization reaction or the reaction conditions, one can produce polymers comprising a wide range of average molecular weights. Under the specified conditions, PEHA polymers with a mean molecular weight between about 100,000 to about 400,000 daltons are produced.

6.2. Methods of Making Stable Delivery Vehicles

6.2.1. Protocol for Formulating DNA/Cationic Polymer Complexes.

PEHA polymer was hydrated in a suitable buffer (for example, 150 mM NaCl; 50 mM NaCl; 10 mM NaCl; 0.2 M dextrose; 50 mM NaCl, 0.2 M dextrose; or 150 mM NaCl, 0.2 M dextrose) at a concentration of about 450 μg of polymer/ml, or up to about 4 mg per ml. DNA (SSV9-pMD-AP) was added to the PEHA polymer at cation/DNA phosphate ratio of between about 1:1 and about 20:1, and incubated for about 10 to 30 minutes. Typically, the concentration and type of salt present during complex formation will vary dependent upon the intended use of the complex (i.e., in vitro versus in vivo).

The resulting cation/DNA complex was either directly applied to cells or is injected into mouse tail veins (I.V.) as a composition comprising about 60 μg of DNA in about 300 μl.

6.2.2. Protocol for Formulating DNA/Cationic Lipid Complexes by Alcohol Evaporation.

A suitable amount of lipofectamine (DOSPA) was evaporated to dryness and dissolved in 40 percent ethanol/250 mM glucose/25 mM NaCl (e.g., enough to provide a DOSPA:DNA nucleotide ratio (mol/mol) of about 0.6). DOPE was added to provide a final mol/mol ratio of DOPE:DOSPA of approximately 62:38.

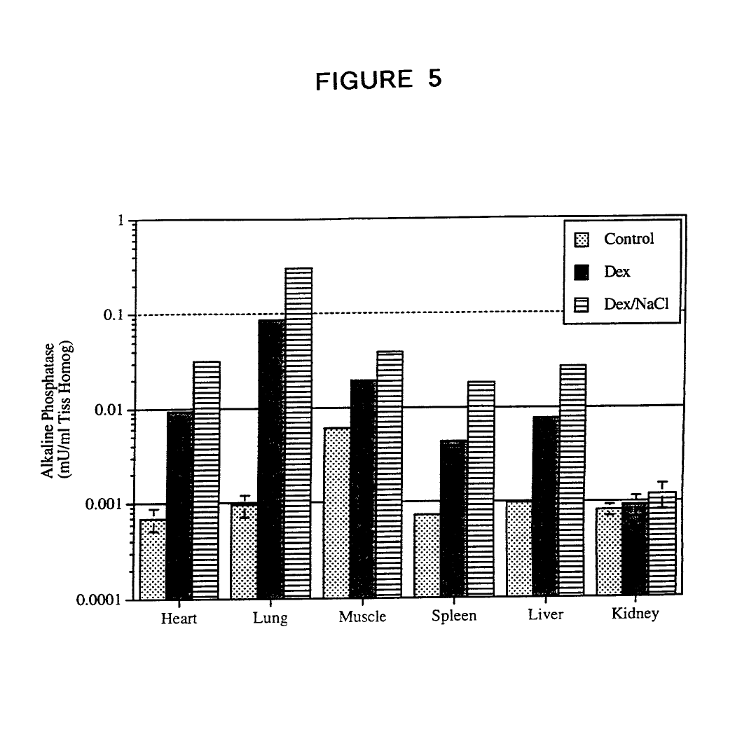

A DNA solution was prepared at a concentration of 1.2 mg/ml in 40 percent ethanol/250 mM glucose/25 mM NaCl. An equal volume of DNA was then added to the lipid solution to yield a final DNA concentration of about 0.63 mg/ml and a final DOSPA/DNA nucleotide ratio of about 0.6. The ethanol/water solution was removed by rotoevaporation which resulted in a thin dry film of DNA/cation complex. The film was then hydrated with water to yield a stable solution of PDVs. FIG. 5 shows in vivo expression data obtained using the PDVs, and, inter alia, compares results obtained using PDVs prepared in the presence or absence of NaCl.

Where TAC was used in lieu of DOSPA, it was used at a TAC:DOPE (mol/mol) ratio of about 75:25, the complexation buffer preferably had a pH of about 6, and the complexation reaction preferably occurred at about room temperature.

6.3. Particle Size Analysis

Particle size analysis was obtained using a Leeds and Northrop laser dynamic light scattering instrument. Characterization of the cationic polymer (in water) showed that particles were formed with a mean size of approximately 200 nm in diameter. The addition of NaCl (150 mM) caused the mean size to increase to about 1,000 nm. The addition of DNA caused the mean size of the particles to decrease to about 400 nm.

PDVs prepared using the alcohol evaporation method typically have a mean diameter of less than 200 nm, and may be extruded to form particles of less than 100 nm mean diameter. In particular, PDVs prepared using TAC may be extruded to yield a mean particle size of between about 40 nm and about 100 nm.

6.4. Cell Transfection With PDVs.

PDVs were formed essentially as described in section 6.2 (150 mM NaCl) and added to approximately 10⁵ NIH 3T3 cells cultured in 0.5 ml of serum free media. After the PDVs (about 10 μg of DNA) were added, the cells were incubated for about 4 hr. The cells were subsequently assayed for expression of the reporter gene by an alkaline phosphatase immunocapture assay. These studies revealed that the PDVs are useful for gene delivery in vitro.

6.5. Use of Polynucleotide Delivery Vehicles In Vivo.

PDVs formed essentially as described in section 6.2 were injected into mice as follows. PDVs comprising approximately 60 μg of DNA were injected into mouse tail veins in a net volume of about 500 μl. Mouse tissue samples were harvested 48 hours after PDV administration and homogenized in buffer at a net concentration of about 100 mg/ml. The homogenates were heated to 65° C. for 30 minutes to inactivate endogenous alkaline phosphatase, and analyzed using an immunocapture assay comprised of adsorbing a secondary antibody to a 96 well plate that binds a subsequently added anti-human placental alkaline phosphatase polyclonal antibody. 0.2 ml of homogenate was added to each well and allowed to incubate overnight at 4° C. The wells were washed, additional 200 μl aliquots were added to the wells and incubated for 2 hours to increase the signal, the wells were washed again, and an alkaline phosphatase substrate was added. The plate was then read using a Molecular Devices plate reader which can determine a Vₘₐₓ for each well. The Vₘₐₓ was converted to mUnits of AP and the data were normalized per 100 mg of tissue using a standard curve ranging from 20 munits to 0.1 mUnits of alkaline phosphatase (AP).

Possibly because of the relatively large size of the PDVs, cardiopulmonary tissues tend to best express genes delivered by PDVs formed by the detergent dialysis method, and delivered as described. By varying the size of the PDVs (by controlling the mean size of the cationic polymers used to construct the PDVs, etc.), the extent and areas of expression may correspondingly vary.

Because the PDV components are inherently biocompatible or have been formulated to have reduced toxicity, animals injected with PDVs display few, if any, signs of overt toxicity in in vivo studies.

Results of the amounts of in vivo expression observed when smaller PDVs were used (produced by the alcohol evaporation method) are shown in FIG. 5. FIG. 5 shows that PDVs can efficiently deliver genes to the heart, lung, muscle, spleen, and liver.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

1. A cationic polymer having the general structure:
. . . (R—X—R)n . . .
wherein R is a cationic group capable of binding nucleic acid, X is a biotolerable covalent cross-linker molecule, and n is number between about ten and up to about ten thousand.

2. The cationic polymer of claim 1 wherein R is a multivalent amine.

3. The cationic polymer of claim 2 wherein said multivalent amine is pentaethylenehexamine.

4. A cationic heteropolymer having the general structure:
( . . . (X—Rₓ—X—R_{y}—X) . . . )
wherein Rₓ is a cationic group that is capable of interacting with nucleic acid; R_{y} is any of a number of cationic groups other then Rₓ that are also capable of interacting with nucleic acid; and X is a biotolerable cross-linker molecule.

5. The cationic heteropolymer of claim 4 wherein at least one of Rₓ or R_{y} is pentaethylenehexamine.

6. A cationic lipid having the general structure:
(R₁, R₂)N—C(O)O—Y
wherein R₁ and R₂ are drawn from the group consisting of H, C₁-C₆ alkyls, alkenyls, or alkynyls, a monovalent amine, or multivalent amine; and Y is a cholesterol or cholesterol derivative.

7. The cationic lipid of claim 6 wherein R₁ and R₂ are monovalent amines.

8. The cationic lipid of claim 7 wherein said monovalent amine is ethylamine.

9. A method of making a polynucleotide delivery vehicle, comprising:
a) complexing a cationic polymer and/or cationic lipid with a polynucleotide in the presence of detergent;
b) removing the detergent whereby a stable polynucleotide delivery vehicle is produced.

10. The method of claim 9 wherein said removing is by dialysis.

11. A method of making a polynucleotide delivery vehicle, comprising:
a) complexing DNA with a cationic polymer and/or cationic lipid in a buffer that substantially maintains the DNA as a B helix;
b) removing the buffer from the complexed DNA; and
c) adding an aqueous solution to the complexed DNA; whereby a polynucleotide delivery vehicle is formed.

12. The method of claim 11 wherein said buffer comprises between about 10 and about 80 percent alcohol.

13. The method of claim 12 wherein said alcohol is ethanol.

14. The method of claim 11 wherein said cationic lipid is the cationic lipid of claim 6.

15. A polynucleotide delivery vehicle produced by the method described by any one of claims 9 through 14.
